(19) United States
(12) Patent Application Publication
(10) Pub. No.: US 2026/0102559 A1
(43) Pub. Date: Apr. 16, 2026

(54) INFUSION PUMP WITH INTEGRATED SET

(71) Applicant: QUASURAS, INC., San Diego, CA (US)

(72) Inventors: David Neese, Escondido, CA (US); Paul M. DiPerna, Escondido, CA (US)

(21) Appl. No.: 19/354,075

(22) Filed: Oct. 9, 2025

Related U.S. Application Data
(60) Provisional application No. 63/708,141, filed on Oct. 16, 2024.

(51) Int. Cl. A61M 5/142; A61M 5/162

(52) U.S. Cl. CPC A61M 5/14248; A61M 5/1626; A61M 2005/1426; A61M 2209/082

(57) ABSTRACT

Rotary microfluidic medical pump and pumping method embodiments are discussed herein that may include elements having an integrated set for establishing fluid communication with a patient's body. Such embodiments may be used for controlled delivery of small and precise volumes of therapeutic or non-therapeutic medical fluids to a patient in a variety of environmental conditions.

### RELATED APPLICATION

The present application claims priority to U.S. Provisional Ser. No. 63/708,141 filed on Oct. 16, 2024 and titled “Infusion Pump with Integrated Set” the entirety of which is incorporated by reference herein.

### BACKGROUND

The delivery of therapeutic and non-therapeutic medical fluids may be commonly performed intravenously (IV) or subcutaneously using an infusion catheter or cannula and a suitable type of pump. For subcutaneous delivery of medical fluids, such as medical fluids used for the treatment of diabetes, pumps may be used in an ambulatory configuration wherein the patient carries the pump with them and an outlet of the pump is disposed in fluid communication with the patient's body through a cannula that has been deployed into the patient's skin or other suitable structure. Typically, for such indications, the pump will be coupled to a separate port or set that includes the cannula with a distal end which is configured to be deployed beneath the patient's skin.

In some cases, this typical arrangement with a pump and a separate set includes multiple components that must be managed and implemented by a patient which may, in some circumstances, be inconvenient and somewhat complicated to operate. What have been needed are simplified pump and set configurations that are user friendly for ambulatory indications.

### SUMMARY

Some embodiments of a medical pump system may include a mount bracket having a base plate and an integral infusion set secured to the base plate. The integral infusion set may include a cannula having an inner lumen and a bracket port in fluid communication with the inner lumen of the cannula. An applicator may be configured to releasably engage the mount bracket and deploy the mount bracket away from the applicator in a motion parallel to a longitudinal axis of the cannula. The applicator may include an applicator driver and an applicator latch configured to releasably engage the mount bracket with the applicator driver in a restrained state. The medical pump system may also include an applicator trigger configured to release the applicator latch and a medical pump including a pump port that is configured to be operatively coupled in fluid communication to the bracket port.

Some embodiments of a medical pump system may include a mount bracket having a base plate and an integral infusion set secured to the base plate which includes a cannula having an inner lumen and a bracket port in fluid communication with the inner lumen of the cannula. The mount bracket may also include a needle cap assembly which is releasably secured in fixed relation to the integral infusion set and which includes a needle extending through the inner lumen of the cannula. The medical pump system may further include an applicator that is configured to releasably engage the mount bracket and deploy the mount bracket away the from the applicator in a motion parallel to a longitudinal axis of the cannula. The applicator may include an applicator driver and an applicator latch configured to releasably engage the mount bracket with the applicator driver in a restrained state. An applicator trigger may be configured to release the applicator latch. A medical pump may also be included in the medical pump system. The medical pump may include a pump port that is configured to be operatively coupled in fluid communication to the bracket port and may be configured to be releasably secured to the base plate.

Some embodiments of a mount bracket for interfacing with a patient may include a base plate and an integral infusion set secured to the base plate within an outer contour of the base plate. The integral infusion set may include a bracket port which is configured to be operatively coupled in fluid communication to a medical pump and a cannula which extends outwardly from a bottom surface of the base plate and which includes an inner lumen disposed in fluid communication with the bracket port. The mount bracket may also include an adhesive layer disposed on the bottom surface of the base plate which is configured to temporarily adhere to the skin of a patient with a distal tip of the cannula disposed beneath an outer surface of the patient's skin.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 is a schematic view in elevation of a mount bracket embodiment of a medical pump system embodiment disposed on a boss of a packaging base covered and sealed by a packaging cover.

FIG. 2 shows the mount bracket of FIG. 1 disposed on the boss of the packaging base with the packaging cover removed.

FIG. 3 illustrates the mount bracket of FIG. 2 with an applicator embodiment in an un-cocked state disposed over the mount bracket with an applicator spring of the applicator operatively coupled to the mount bracket.

FIG. 4 illustrates the applicator and mount bracket of FIG. 3 with the applicator having been depressed as indicated by the downward arrow of FIG. 3 allowing the walls of the applicator to move downward around the mount bracket and boss until the applicator is in a cocked state with stored energy in the applicator spring and an applicator latch securing the applicator to the mount bracket with the applicator in the cocked state.

FIG. 5 illustrates the cocked applicator and mount bracket of FIG. 4 having been disposed on a surface of a patient's skin at a suitable deployment site with the walls of the applicator in contact with the patient's skin and the mount bracket, including a cannula and associated needle of an integrated infusion set thereof, disposed above and spaced from the patient's skin in a pre-deployment configuration.

FIG. 5A is an enlarged view of the encircled portion 5A-5A of FIG. 5.

FIG. 6 illustrates the mount bracket and applicator of FIG. 5 after actuation of the applicator trigger as indicated by the downward arrow of FIG. 5 and subsequent downward motion of the mount bracket as indicated by the second downward arrow of FIG. 5 resulting in a configuration with a bottom surface of the mount bracket disposed in contact with the patient's skin and the cannula and needle of the integrated infusion set of the mount bracket having penetrated to a position beneath the patient's skin.

FIG. 7 shows the mount bracket deployed onto the patient's skin with the applicator having been removed from the mount bracket as indicated by the upward arrow of FIG. 6 and with the cannula and associated needle disposed within and extending distally from an inner lumen of the cannula.

FIG. 8 shows the mount bracket disposed on the patient's skin with the needle removed from a port of the of the integrated infusion set of the mount bracket and the inner lumen of the cannula by withdrawal of the needle and associated needle cap as indicated by the upward arrow of FIG. 8 leaving the cannula disposed in fluid communication with the patient's tissue beneath the patient's skin.

FIG. 9 shows the mount bracket and cannula thereof in operative communication with a medical pump that has been coupled to the mount bracket (as indicated by the downward arrow in FIG. 9) with a pump port thereof in fluid communication with the bracket port in a configuration suitable for delivery of therapeutic fluid from the medical pump to the patient's subdermal tissue.

FIG. 10 illustrates an enlarged view of the pump port of FIG. 9 coupled to the bracket port and illustrating a needle of the pump port disposed through a septum of the bracket port providing sealed fluid communication between an inner lumen of the needle of the pump port and the inner lumen of the cannula of the mount bracket.

FIG. 11 is a schematic view in elevation of a mount bracket embodiment of an infusion pump system embodiment disposed on a boss of a packaging base covered and sealed by a packaging cover.

FIG. 12 shows the mount bracket of FIG. 11 disposed on the boss of the packaging base with the packaging cover removed.

FIG. 13 illustrates the mount bracket of FIG. 12 with an applicator embodiment in an un-cocked state disposed over the mount bracket.

FIG. 14 illustrates the applicator and mount bracket of FIG. 13 with the applicator having been depressed as indicated by the downward arrow of FIG. 13 allowing the walls of the applicator to move downward around the mount bracket and boss until the applicator is in cocked state with stored energy in the applicator spring and an applicator latch securing the applicator to the mount bracket with the applicator in the cocked state.

FIG. 14A illustrates the applicator and mounting bracket in longitudinal section.

FIG. 15 illustrates the cocked applicator and mount bracket of FIG. 14 having been disposed on a surface of a patient's skin at a suitable deployment site with the walls of the applicator in contact with the patient's skin and the mount bracket, including a cannula and associated needle of an integrated infusion set thereof, disposed above and spaced from the patient's skin in a pre-deployment configuration.

FIG. 15A shows the cocked applicator and mounting bracket embodiments of FIG. 15 in longitudinal section.

FIG. 15B is an enlarged view of the encircled portion 15B-15B of FIG. 15.

FIG. 16 illustrates the mount bracket and applicator of FIG. 15 after actuation of the applicator trigger as indicated by the lateral arrow of FIG. 15A and subsequent downward motion of the mount bracket as indicated by the second downward arrow of FIG. 15A resulting in a configuration with a bottom surface of the mount bracket disposed in contact with the patient's skin and the cannula and needle of the integrated infusion set of the mount bracket having penetrated to a position beneath the patient's skin.

FIG. 17 shows the mount bracket deployed onto the patient's skin with the applicator having been removed from the mount bracket as indicated by the upward arrow of FIG. 16 and with the cannula and associated needle disposed within and extending distally from an inner lumen of the cannula.

FIG. 17A is a perspective view of the mount bracket and needle cap assembly of FIG. 17.

FIG. 17AA is a perspective view of the mount bracket and needle cap assembly of FIG. 17.

FIG. 17AAA is an enlarged view of the encircled portion 17AAA-17AAA of FIG. 17AA.

FIG. 17 B shows the mount bracket of FIG. 17A with the needle cap assembly detached from the mount bracket.

FIG. 18 shows the mount bracket disposed on the patient's skin with the needle removed from a port of the of the integrated infusion set of the mount bracket and the inner lumen of the cannula by withdrawal of the needle and associated needle cap assembly as indicated by the upward arrow of FIG. 18 leaving the cannula disposed in fluid communication with the patient's tissue beneath the patient's skin surface.

FIG. 18A is an exploded perspective view of a two component medical pump system embodiment and associated mount bracket embodiment.

FIG. 18B is a perspective view of the medical pump system embodiment and mount bracket embodiment of FIG. 18A with the two components of the medical pump system coupled together and disposed above the mount bracket.

FIG. 19 shows the mount bracket and cannula thereof in operative communication with a medical pump that has been coupled to the mount bracket (as indicated by the downward arrow in FIG. 19) with a pump port thereof in fluid communication with the bracket port in a configuration suitable for delivery of therapeutic fluid from the medical pump to the patient's subdermal tissue.

FIG. 20 illustrates an enlarged view of the pump port of FIG. 19 coupled to the bracket port and illustrating a hollow needle of the pump port disposed through a septum of the bracket port providing sealed fluid communication between an inner lumen of the needle of the pump port and the inner lumen of the cannula of the mount bracket.

The drawings are intended to illustrate certain exemplary embodiments and are not limiting. For clarity and ease of illustration, the drawings may not be made to scale, and in some instances, various aspects may be shown exaggerated or enlarged to facilitate an understanding of particular embodiments.

### DETAILED DESCRIPTION

Delivery of therapeutic fluids for treatment of a patient or any other suitable purpose may commonly be performed intravenously (IV) or subcutaneously using systems that include pumps such as syringe pumps, peristaltic pumps as well as others. However, these types of pumps do not always perform consistently and cost effectively, particularly when used in varying environmental conditions. Medical pump system embodiments and related components that address issues such as these are discussed in U.S. patent application Ser. No. 16/028,256, filed Jul. 5, 2018, by P. DiPerna et al., titled “Medical Pump with Flow Control”, U.S. patent application Ser. No. 16/520,521, filed Jul. 24, 2019, by P. DiPerna et al., titled “Subcutaneous Access Hub with Multiple Cannula Ports”, and U.S. patent application Ser. No. 15/122,132, Publication No. US 2016/0361489 A1, filed Mar. 3, 2015, by P. DiPerna, titled “Fluid Delivery Pump”, each of which is incorporated by reference herein in its entirety.

For medical pump system embodiments that are configured to deliver therapeutic fluids subcutaneously, and particularly those that may be used by a patient end user, it may be desirable to maximize the simplicity and user friendliness of the deployment of such systems. It may also be desirable to keep an outer profile of such pump system embodiments as small as possible when in a deployed state upon a patient's body. Many medical pump systems currently in use utilize infusion sets in order to establish fluid communication between a medical pump system and a subdermal site within a patient's body. Infusion sets are typically small assemblies containing a flexible or rigid hollow cannula with a distal end and output port thereof disposed subcutaneously in a patient's skin which allows therapeutic fluid from the medical pump system to pass into the subcutaneous space once the medical pump system is coupled in fluid communication with the hollow cannula.

Such infusion sets may include flexible tubing having an inner lumen which is in fluid communication with the inner lumen of the hollow cannula at one end and an output port of the medical pump assembly at the other end. The hollow cannula of the typical infusion set may be secured to a housing or hub type structure that includes an adhesive surface which is configured to temporarily adhere to a patient's skin while holding the hub and associated cannula in place with the distal end of cannula disposed subcutaneously. Such a typical configuration of medical pump system and infusion set includes multiple components that are disposed on multiple positions on the patient's body. That is, the adhesive surface of the hub of the infusion set will be secured to the patient's body at one position and the medical pump system may be secured to the patient's body at another position near the infusion set hub.

Embodiments of medical pump systems and components thereof discussed herein propose to include an integral infusion set into the medical pump system, or components thereof, such that the infusion set and medical pump system are combined so as to occupy the same or substantially the same location on the patient's body once deployed for use. In addition, for some embodiments, such medical pump systems simplify the deployment process by allowing the end user to deploy both the medical pump system and infusion set at the same time.

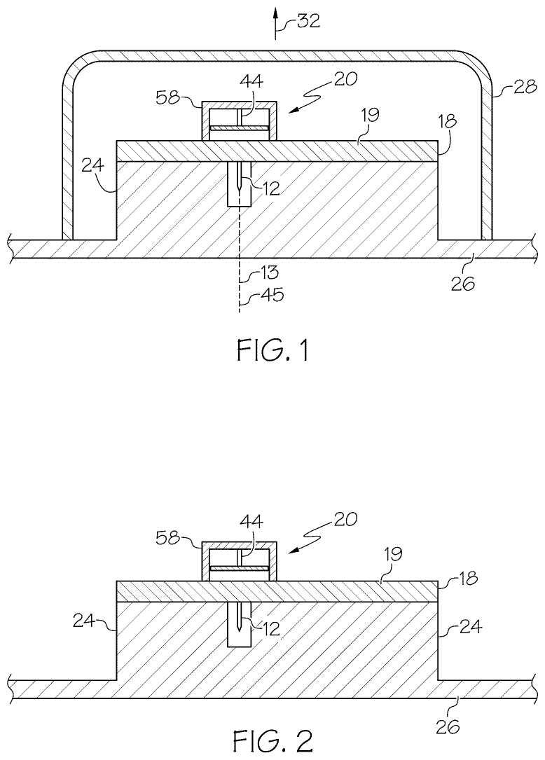

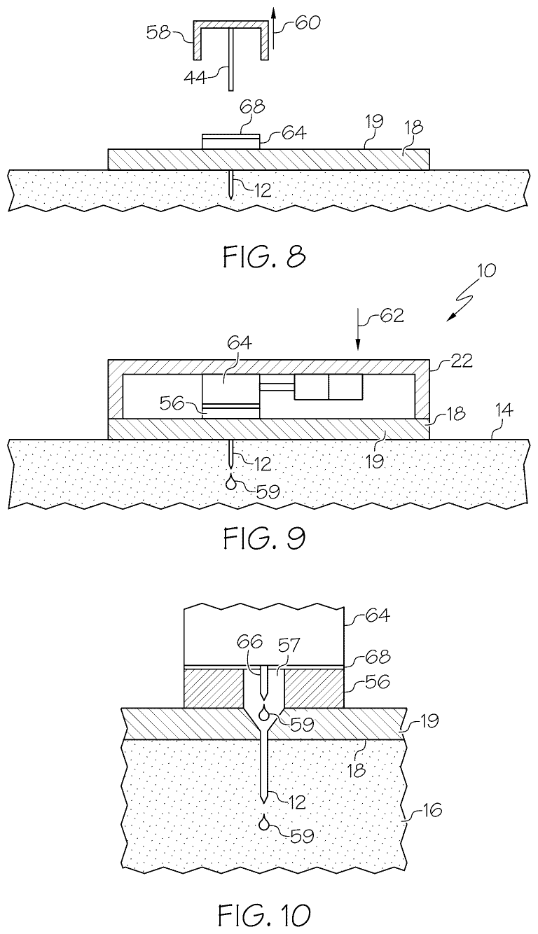

FIGS. 1-10 illustrate schematically an embodiment of a medical pump system 10 (see FIG. 9) and components thereof which are configured to simplify deployment of a cannula 12 of the system 10 into the skin 14 of a patient 16 and, in some cases, reduce the footprint of the medical pump system 10 on the patient's body. For the embodiment shown, the medical pump system 10 includes a mount bracket 18 with an integral infusion set 20 that is configured to automatically couple in fluid communication to an output port of a medical pump 22 of the medical pump system 10 when the medical pump 22 is connected to the mount bracket 18. For some such embodiments, a base plate 19 of the mount bracket 18 may include a thin plate of lightweight and rigid material with a top surface that is generally parallel to a bottom surface of the base plate structure 19. The cannula 12 may have a generally straight configuration with a longitudinal axis 13.

The mount bracket 18 may have an outer peripheral contour and footprint which is the same as or similar to the outer peripheral contour and footprint of the medical pump 22. In such cases, the mount bracket 18 may be disposed between an outer surface of the patient's skin 14 and a bottom surface of the medical pump 22 once the system 10 is deployed onto the patient 16. For some embodiments, the entire outer peripheral contour of the mount bracket 18 may be disposed between the bottom surface of the medical pump 22 and the patient's skin 14 when the system 10 is deployed. For some embodiments, portions of the outer peripheral contour of the mount bracket 18 may extend laterally outside of the outer peripheral contour of the medical pump 22 when the system is deployed. Typically, the integral infusion set 20 of the mount bracket 18 will be disposed within the outer peripheral contour of the medical pump 22 when the system is deployed onto the patient's skin 14.

FIG. 1 shows the mount bracket embodiment 18 of the medical pump system 10 embodiment disposed on a boss 24 of a packaging base 26 covered and sealed by a packaging cover 28. In some cases, the bottom surface of the mount bracket 18 will be releasably secured to a top surface of the boss 24 in order to hold the mount bracket 18 in place prior to use. FIG. 2 shows the mount bracket 18 of FIG. 1 disposed on the boss 24 of the packaging base 26 with the packaging cover 28 removed from the base 26 as indicated by the arrow 32 in FIG. 1. Such a packaging cover 24 may be sealed to the packaging base 26 to provide an airtight sterile inner volume for the mount bracket 18 prior to use.

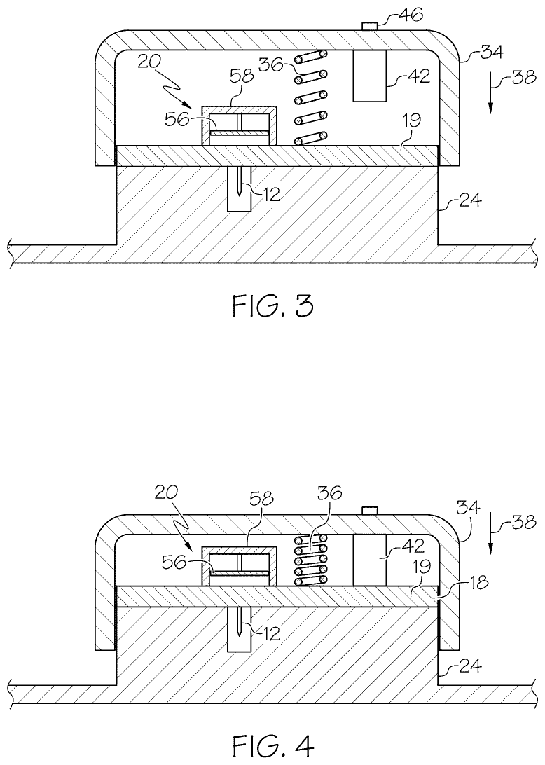

FIG. 3 illustrates the mount bracket 18 with an applicator embodiment 34 in an un-cocked state disposed over the mount bracket 18 with an applicator driver in the form of an applicator spring 36 of the applicator 34 operatively coupled to the mount bracket 18. The applicator driver 36 is configured to impart kinetic energy to the mount bracket 18 along a longitudinal axis 13 of the cannula 12 and/or along a longitudinal axis 45 of an optional needle 44 discussed below. The applicator driver 36 may include any suitable configuration such as a biasing element in the form of a spring such as a coil spring, leaf spring, gas piston spring or the like. For some embodiments, the outer peripheral contour of the boss 24 may be the same as or similar to the outer peripheral contour of the bottom surface of the mount bracket 18 with a profile of the top surface of the boss 24 also being the same as or similar to that of the bottom surface of the mount bracket 18. Such a configuration may useful for holding the mount bracket 18 in place within the packaging while still allowing the mount bracket 18 and boss 24 to extend upwardly into a cavity of a cooperating deployment applicator 34 or the like without unwanted mechanical interference therebetween.

FIG. 4 illustrates the applicator 34 and mount bracket 18 of FIG. 3 with the applicator 34 having been depressed as indicated by the downward arrow 38 of FIG. 3 allowing the walls of the applicator 34 to move downward around the mount bracket 18 and boss 24 until the applicator 34 is in a cocked state. In the cocked state of the applicator 34 there is stored energy in the applicator spring 36 with an applicator latch 42 securing the applicator 34 in fixed but releasable relation to the mount bracket 18. For some embodiments, once the applicator 34 is suitable aligned with the mount bracket 18 as shown in FIG. 3, the applicator 34 may be pressed down onto the mount bracket 18 and boss 24 by a user until a click or the like is heard or felt by the user indicating that the applicator latch 42 has latched and is securing the applicator 34 to the mount bracket 18 in the cocked state ready for deployment.

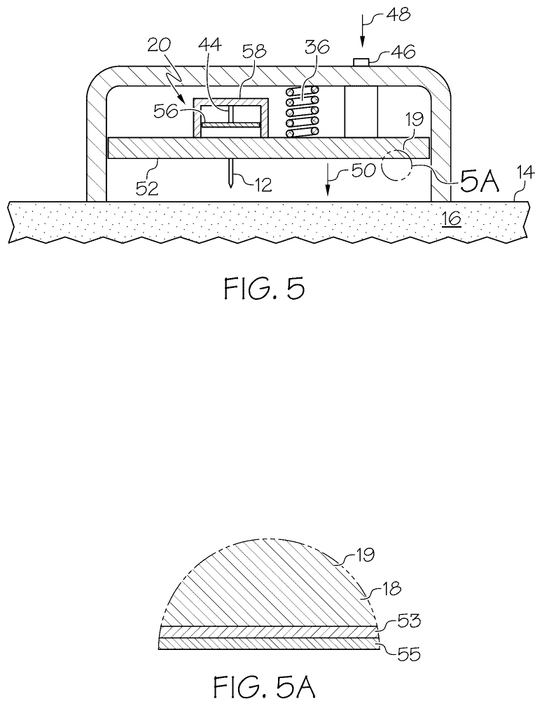

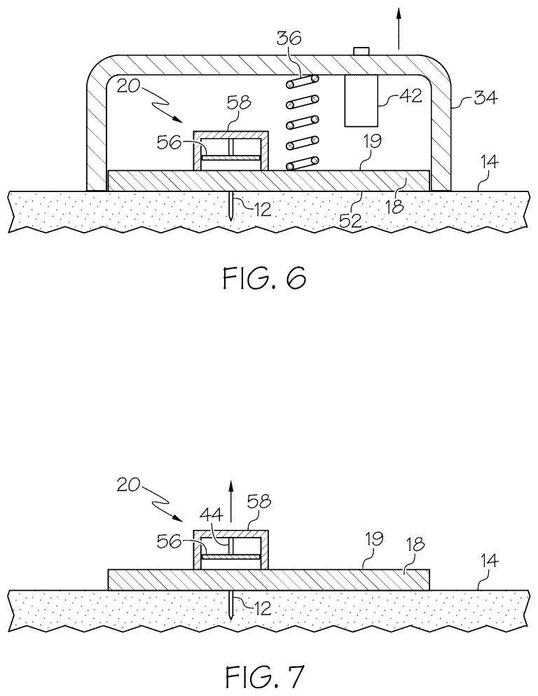

FIG. 5 illustrates the cocked applicator 34 and mount bracket 18 of FIG. 4 having been disposed on a surface of a patient's skin 14 at a suitable deployment site with the walls of the applicator 34 in contact with the patient's skin 14 and the mount bracket 18, including the cannula 12 and an associated needle 44 (see FIG. 8) of the integrated infusion set 20 thereof, disposed above and spaced from the patient's skin 14 in a pre-deployment configuration. FIG. 6 illustrates the mount bracket 18 and applicator 34 of FIG. 5 after actuation of the applicator trigger 46 as indicated by the downward arrow 48 of FIG. 5 and subsequent downward motion of the mount bracket 18 along a longitudinal axis 13 of the cannula as indicated by the second downward arrow 50 of FIG. 5. Such actuation and displacement of the mount bracket 18 results in a configuration with a bottom surface 52 of the mount bracket 18 disposed in contact with the patient's skin 14 and the cannula 12 and needle 44 of the integrated infusion set of the mount bracket 18 having penetrated to a position beneath the patient's skin 14.

In some cases, the bottom surface 52 of the mount bracket 18 may include an adhesive layer 53 that is configured to temporarily adhere to an outside surface of the patient's skin 14 (see FIG. 5A). For some such embodiments, the adhesive layer 53 may be covered with a peel away type covering 55 that is removed just prior to actuation of the applicator trigger 46 and subsequent actuation of the applicator 34 and contact of the adhesive layer with the patient's skin 14. For some embodiments, the upper surface of the boss 24 of the packaging base may serve as the covering of the adhesive layer or any other such suitable configuration such that as the mount bracket 18 is removed from the boss 24, the adhesive layer 53 on the bottom surface 52 of the mount bracket 18 becomes exposed and ready to adhere to the outer surface of the patient's skin 14. Such a configuration may save the end user from the additional step of removing a separate covering 55 thus making such an embodiment more user friendly and convenient.

In some instances, it may be desirable to include vent holes or fenestrations in the body of the base plate 19 of the mount bracket 18 in order to increase deployment velocity and reduce air drag and air entrapment/buffering between the bottom surface 52 of the mount bracket and the outside surface of the patient's skin during rapid downward translation of the mount bracket 18 towards the patient's skin 14 during deployment. It may also be desirable to minimize the weight of the mount bracket 18 overall in order to achieve a desired deployment velocity of the mount bracket 18 onto and into the patient's skin 14.

For some embodiments, the bottom surface 52 of the mount bracket 18 may be flat or substantially flat in profile. For some embodiments, the bottom surface 52 of the mount bracket may have a curved profile in order to better conform to certain outer profiles of the patient's anatomy at desired deployment sites. In some cases, the bottom surface of the mount bracket 18 may have a generally concave profile with a radius of curvature of about 50 mm to about 200 mm.

FIG. 7 shows the mount bracket 18 deployed onto the patient's skin 14 with the applicator 34 having been removed from the mount bracket 18 as indicated by the upward arrow 54 of FIG. 6 and with the cannula 12 and associated needle 44 disposed within and extending distally from an inner lumen of the cannula 12. Thereafter, the needle 44 may be removed from the inner lumen of the cannula 12 by proximally withdrawing the needle 44 by gripping the needle cap 58 and proximally withdrawing the needle cap 58 upward and away from a bracket port 56 of the mount bracket 18. In some cases, the needle cap 58 may be releasably secured to the mount bracket 18 including any suitable portion thereof such as the bracket port 56. FIG. 8 shows the mount bracket 18 disposed on the patient's skin 14 with the needle 44 removed from the inner lumen of the cannula and the bracket port 56 of the of the integrated infusion set of the mount bracket 18. The withdrawal of the needle 44 and associated needle cap 58 is indicated by the upward arrow 60 of FIG. 8. Withdrawal of the needle 44 and associated needle cap 58 leaves the bracket port 56, and, in particular, the inner lumen of the cannula 12 disposed in fluid communication with the patient's tissue beneath the patient's skin 14. In some instances, the cannula may extend from the bottom surface 52 of the mount bracket 18 by a length of about 4 mm to about 10 mm, more specifically, about 5 mm to about 7 mm.

It should be noted that in some cases the needle 44 may be configured to provide the cannula 12 with a rigid tissue penetrating core structure so that the hollow cannula 12 may be made, in some instances, from a soft compliant material such as a polymer or the like which may be more comfortable for the patient after deployment and during use. The needle 44 may thus be made from a high strength rigid material such as stainless steel, nitinol or the like and may include a sharpened distal tip which is configured for tissue penetration. The needle 44 may also have a generally straight configuration to facilitate tissue penetration in a linear motion along a longitudinal axis 45 thereof. In some cases, the sharpened distal tip of the needle 44 may include a trocar like faceted configuration. For some embodiments, the needle 44 may also be configured to automatically withdraw into a needle chamber (not shown) of the needle cap 58 upon withdrawal of the needle 44 from the cannula 12 and withdrawal of the needle cap 58 from the mount bracket 18. Such a configuration may be useful in order to protect the end user from the sharpened distal tip of the needle 44 and facilitate disposal of the needle cap 58 and needle 44 assembly after use.

FIG. 9 shows the mount bracket 18 and cannula 12 thereof operatively coupled to the infusion pump assembly 22. The infusion pump assembly 22 has been coupled to the mount bracket 18 (as indicated by the downward arrow 62 in FIG. 9) with a pump port 64 of the infusion pump assembly 22 operatively coupled to the bracket port 56 such that the pump port 64 is in fluid communication with the bracket port 56 in a configuration suitable for delivery of therapeutic fluid 59 from the infusion pump assembly 22 to the patient's body 16 which may include the patient's skin 14 as well as subdermal tissue beneath the patient's skin. For some embodiments, the infusion pump assembly 22 may be operatively coupled to the mount bracket 18 by merely pressing the infusion pump assembly 22 down onto the mount bracket 18. For some such embodiments, the infusion pump assembly 22 and mount bracket 18 may be configured to mechanically couple together during such an approximation (such as by a snap fit or the like) and also automatically couple the pump port 64 to the bracket port 56 in fluid communication with each other as the needle 66 penetrates the septum 68 during the same simple movement. Such a configuration reduces the number of components required to be managed by the end user and simplifies the deployment process of the medical pump system 10 overall.

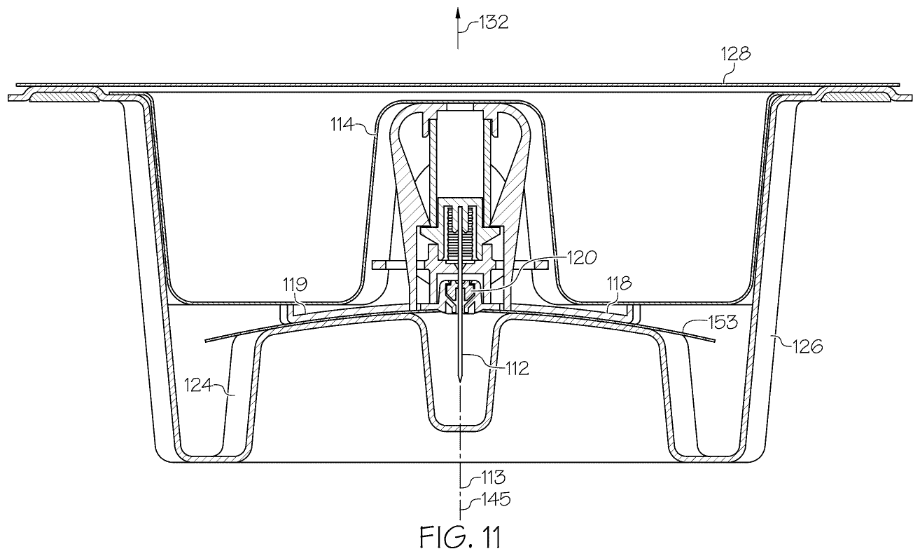

FIG. 10 illustrates an enlarged view of the pump port 64 coupled to the bracket port 56 and showing a hollow needle 66 of the pump port 64 disposed through a septum 68 of the bracket port 56 providing sealed fluid communication between an inner lumen of the hollow needle 66 of the pump port 64 and the inner lumen of the cannula 12 of the mount bracket 18 which allows a sealed flow of therapeutic fluid 59 from the pump chamber (not shown) of the infusion pump assembly 22 through the hollow needle 66 of the pump port 64 and into an interior volume 57 of the bracket port 56 and thereafter through the inner lumen of the cannula 12 and then out a distal port of the cannula 12 and into a subdermal region of the patient's tissue 16 as shown in FIG. 10. In some cases, the cannula 12 may include a hollow tubular structure that may be made from a flexible material including polymers such as polyurethane, nylon, PTFE or the like. In some cases, the cannula 12 may also be made from a biocompatible high strength rigid material such as stainless steel, nitinol or the like.

Once the use of an initial infusion pump assembly 22 has been completed, the initial infusion pump assembly 22 may be removed from the mount bracket 18 with the septum of the bracket port 56 self-sealing as the hollow needle 66 of the pump port 64 is withdrawn therefrom. Thereafter, a new infusion pump assembly 22 may be operatively coupled to the mount bracket 18 as described above or the mount bracket may be removed from the patient 16 by mechanically pulling the mount bracket 18 upwards away from the outer surface of the patient's skin 14. As the mount bracket 18 is removed from the patient, the cannula 12 will contemporaneously be withdrawn from the patient's body 16 and the delivery channel in the patient's skin 14 allowed to heal. In cases where the bottom surface 52 of the mount bracket 18 includes an adhesive layer, removal of the mount bracket 18 from the outer surface of the patient's skin 14 will require a breaking of the adhesive bond between the adhesive layer and the outer surface of the patient's skin 14. In addition to switching out a new infusion pump assembly 22 for an a previously deployed infusion pump assembly 22, a deployed infusion pump assembly 22 may be temporarily removed from the mount bracket 18 as noted above and then re-deployed onto the mount bracket 18 at a later time. Such temporary removal of a deployed infusion pump assembly 22 may be carried out to facilitate activities of a user such as swimming, bathing or the like.

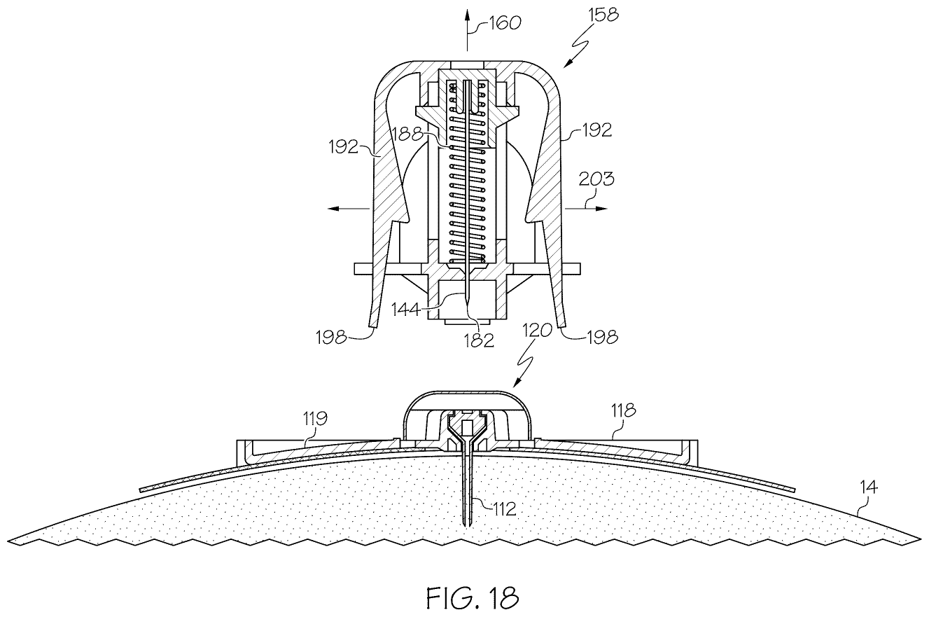

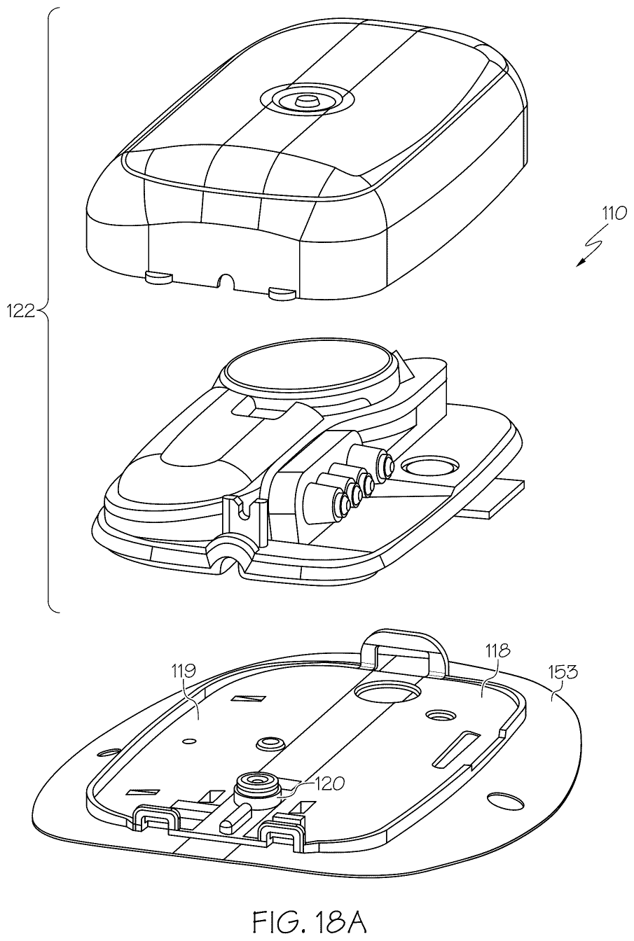

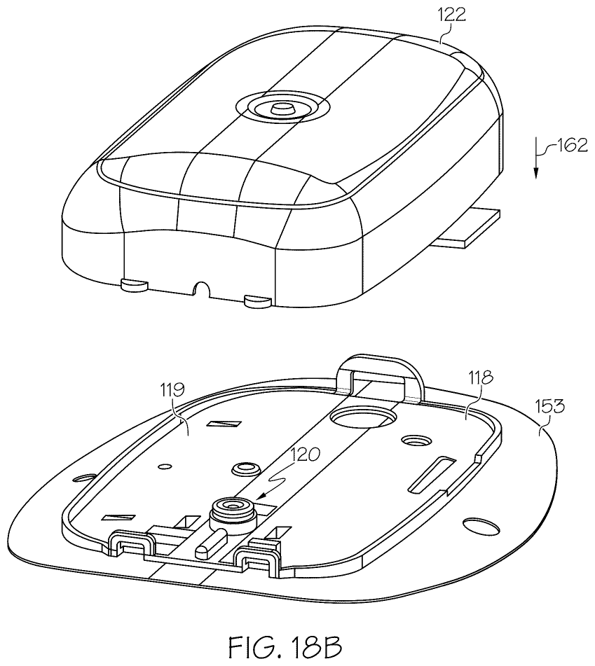

FIGS. 11-20 illustrate another embodiment of a medical pump system 110 (see FIG. 18A) and components thereof which are configured to simplify deployment of a cannula 112 of the system 110 into patient's body 16 such as the skin 14 and, in some cases, reduce the footprint of the medical pump system 110 on the patient's body 16. For the embodiment shown, the medical pump system 110 includes a mount bracket 118 with an integral infusion set 120 that is configured to automatically couple in fluid communication to an output port of a medical pump 122 of the medical pump system 110 when the medical pump 122 is releasably secured or otherwise connected to the mount bracket 118. For some such embodiments, a base plate 119 of the mount bracket 118 may include a thin plate of lightweight and rigid material with a top surface that is generally parallel to a bottom surface of the base plate structure 119. The cannula 112 may have a generally straight configuration with a longitudinal axis 113 that may be perpendicular to an adjacent bottom surface of the base plate 119.

In some cases, the mount bracket 118 may have an outer peripheral contour and footprint which is the same as or similar to the outer peripheral contour and footprint of the medical pump 122. In such cases, the mount bracket 118 may be disposed between an outer surface of the patient's skin 14 and a bottom surface of the medical pump 122 once the system 110 is deployed onto the patient 16. For some embodiments, the entire outer peripheral contour of the mount bracket 118 may be disposed between the bottom surface of the medical pump 122 and the outer surface of the patient's skin 14 when the system 110 is deployed. For some embodiments, portions of the outer peripheral contour of the mount bracket 118 may extend laterally outside of the outer peripheral contour of the medical pump 122 when the system is deployed. Typically, the integral infusion set 120 of the mount bracket 118 will be disposed within the outer peripheral contour of the medical pump 122 and outer peripheral contour of the base plate 119 when the system is deployed onto the patient's skin 14.

FIG. 11 shows the mount bracket embodiment 118 of the medical pump system 110 embodiment disposed on a boss 124 of a packaging base 126 covered and sealed by a packaging cover 128. In some cases, the bottom surface of the mount bracket 118 will be releasably secured to a top surface of the boss 124 in order to hold the mount bracket 118 in place prior to use. For some embodiments, the bottom surface of the base plate 119 of the mount bracket 118 may be releasably secured to the top surface of the boss 124 with an adhesive layer that remains with the mount bracket 118 while the mount bracket 118 is being withdrawn or otherwise pulled away from the top surface of the boss 124. The adhesive layer may be configured to be temporarily secured to an outside surface of the patient's skin 14 after removal from the top surface of the boss 124. A secondary molded tray 114 may be disposed between the mount bracket 118 and the packaging cover 128 in some cases in order to stabilize the position of the mount bracket 118 within the packaging base 126. Such a secondary tray 114 may also include one or more molded pockets or recesses that may be used to secure other elements or components of the medical pump system 110 such as a fluid cartridge or actuator of some medical pump system embodiments 110.

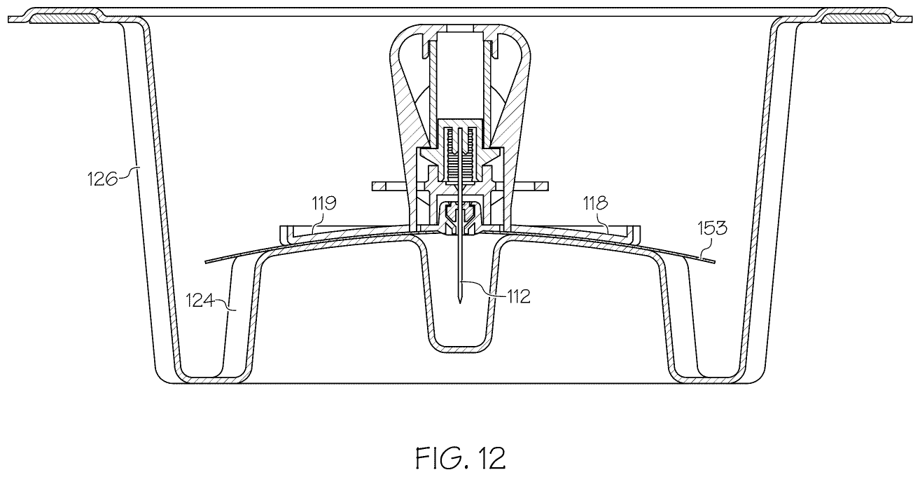

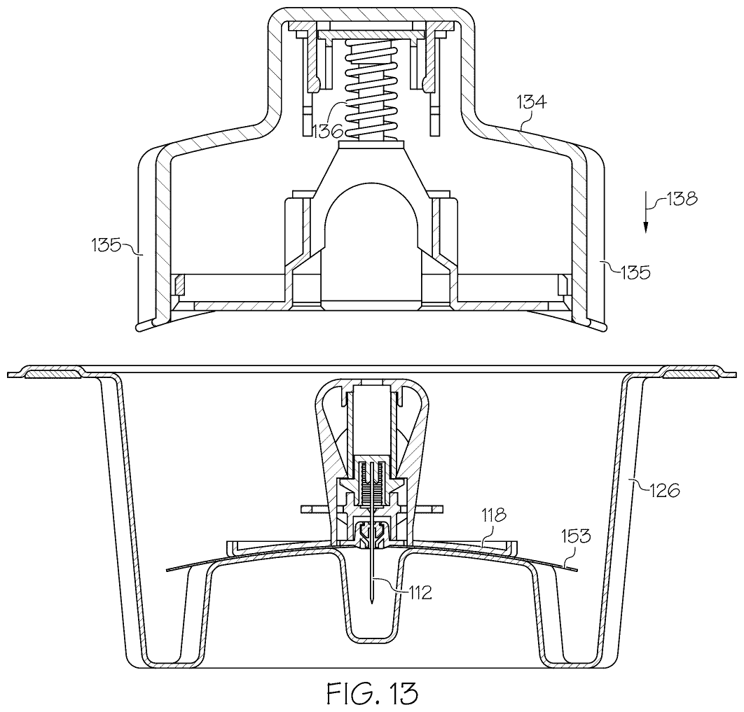

FIG. 12 shows the mount bracket 118 of FIG. 11 disposed on the boss 124 of the packaging base 126 with the packaging cover 128 removed from the base 126 as indicated by the arrow 132 in FIG. 11. Such a packaging cover 124 may be sealed to the packaging base 126 to provide an airtight sterile inner volume for the mount bracket 118 prior to use. FIG. 13 illustrates the mount bracket 118 with an applicator embodiment 134 in an un-cocked state disposed over the mount bracket 118 with an applicator driver in the form of an applicator spring 136 of the applicator 134 positioned to be operatively coupled to the mount bracket 118. The applicator driver 136 when released from a cocked constrained state is configured to impart kinetic energy to the mount bracket 118 via translation along a longitudinal axis 113 of the cannula 112 and/or along a longitudinal axis 145 of an optional needle 144 discussed below. The applicator driver 136 may include any suitable configuration such as a biasing element in the form of a spring such as a coil spring, leaf spring, gas piston spring or the like.

For some embodiments, the outer peripheral contour of the boss 124 may be the same as or similar to the outer peripheral contour of the bottom surface of the mount bracket 118 with a profile of the top surface of the boss 124 also being the same as or similar to that of the bottom surface of the mount bracket 118. Such a configuration may useful for holding the mount bracket 118 in place within the packaging while still allowing the mount bracket 118 and boss 124 to extend upwardly into a cavity of a cooperating deployment applicator 134 or the like without unwanted mechanical interference therebetween.

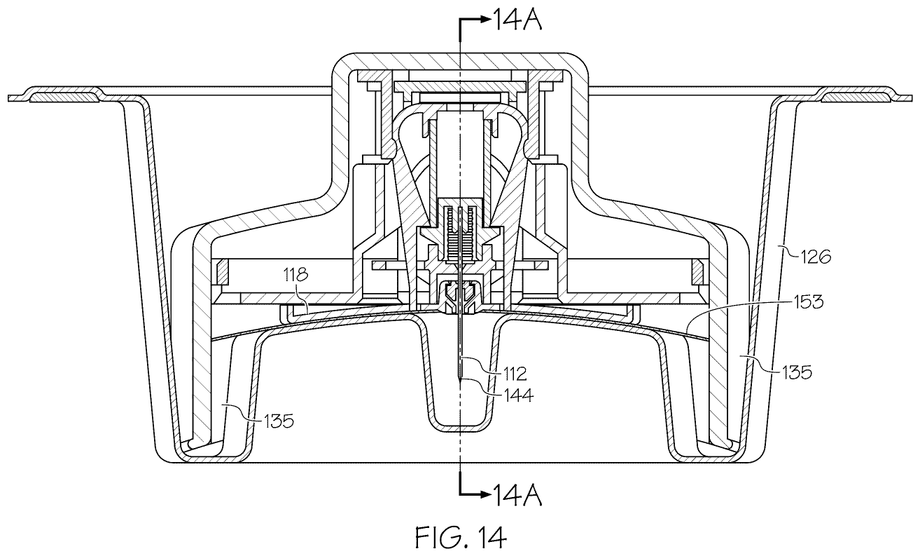

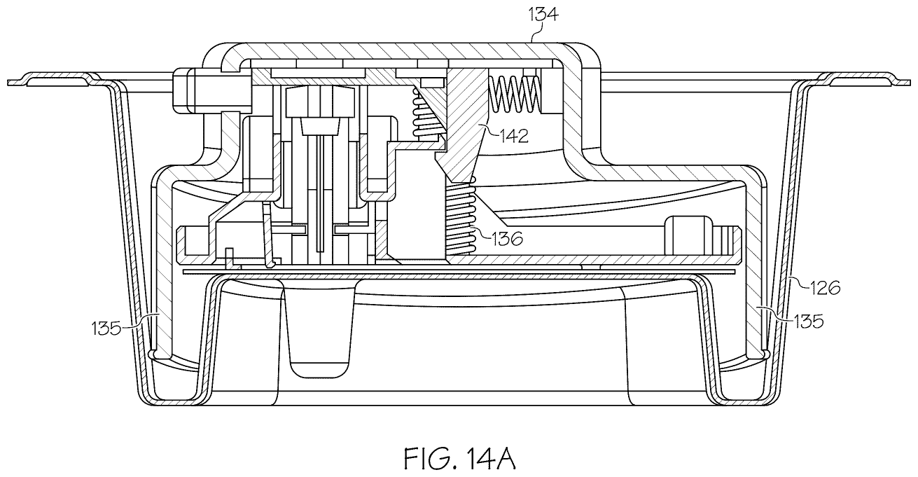

FIGS. 14 and 14A illustrate the applicator 134 and mount bracket 118 of FIG. 13 with the applicator 134 having been depressed as indicated by the downward arrow 138 of FIG. 13 allowing the walls 135 of the applicator 134 to move downward around the mount bracket 118 and boss 124 until the applicator 134 is in a cocked state with an applicator driver in the form of the applicator spring 136 compressed in order to store kinetic energy.

In the cocked state of the applicator 134, there is stored energy in the compressed applicator spring 136 with an applicator latch 142 securing the applicator 134 in fixed but releasable relation to the mount bracket 118. For some embodiments, once the applicator 134 is suitable aligned with the mount bracket 118 as shown in FIG. 13, the applicator 134 may be pressed down onto the mount bracket 118 and boss 124 by a user until a click or the like is heard or felt by the user indicating that the applicator latch 142 has latched and is securing the applicator 134 to the mount bracket 118 in the cocked state ready for deployment.

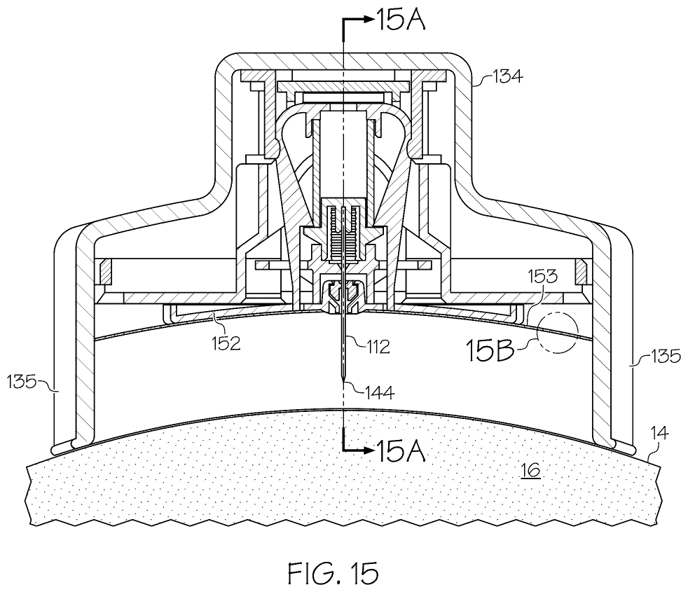

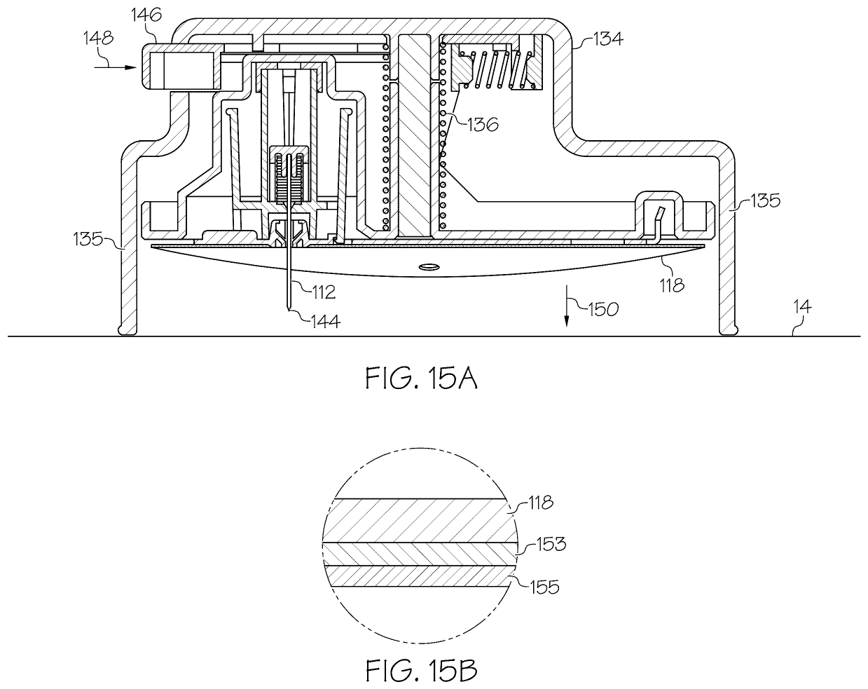

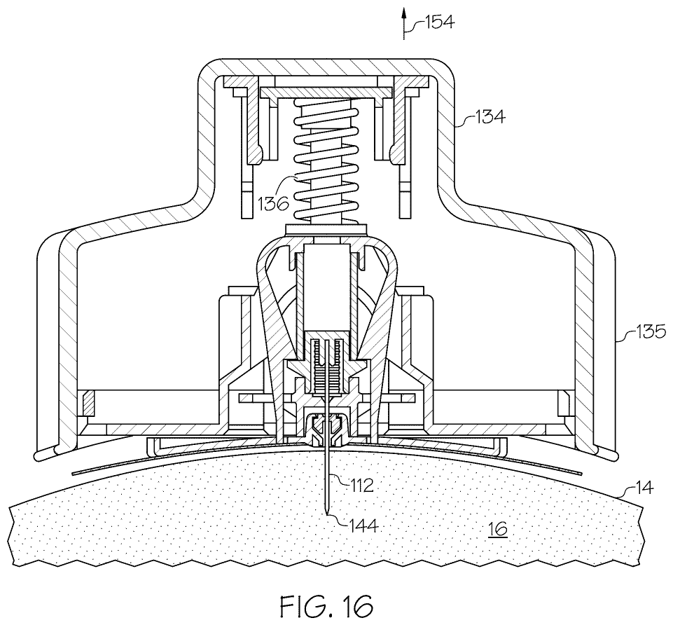

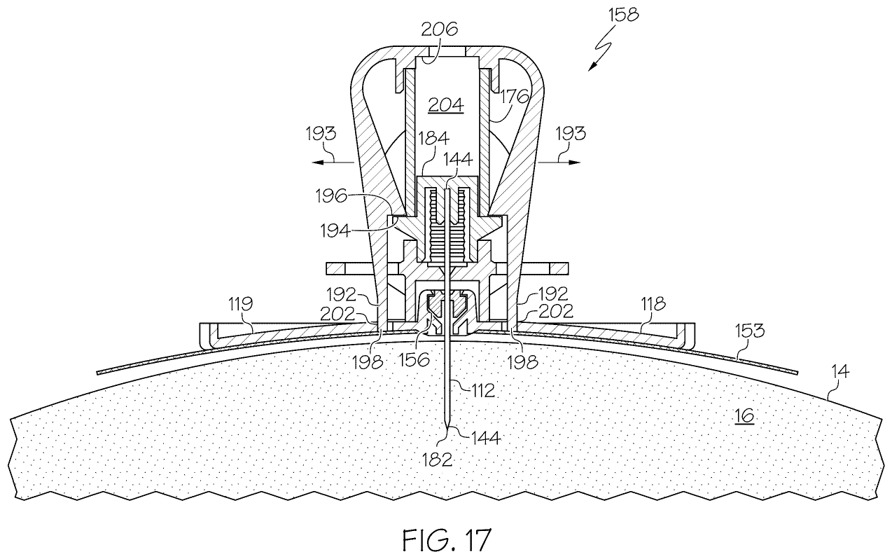

FIGS. 15 and 15A illustrate the cocked applicator 134 and mount bracket 118 of FIG. 14 having been disposed on a surface of a patient's skin 14 at a suitable deployment site with the walls of the applicator 134 in contact with the patient's skin 14 and the mount bracket 118, including the cannula 112 and an associated needle 144 of the integrated infusion set 120 thereof, disposed above and spaced from the outer surface of the patient's skin 14 in a pre-deployment configuration. FIG. 16 illustrates the mount bracket 118 and applicator 134 of FIG. 15 after actuation of the applicator trigger 146 as indicated by the downward arrow 148 of FIG. 15 and subsequent downward motion of the mount bracket 118 as indicated by the second downward arrow 150 of FIG. 15 resulting in a configuration with a bottom surface 152 of the mount bracket 118 disposed in contact with an outer surface of the patient's skin 14 and the cannula 112 and needle 144 of the integrated infusion set of the mount bracket 118 having penetrated to a position beneath the outer surface of the patient's skin 14 as shown in FIG. 16.

In some cases, the bottom surface 152 of the mount bracket 118 may include an adhesive layer 153 that may extend laterally beyond an outer perimeter of the base plate 119 of the mount bracket 118 that is configured to temporarily adhere to an outside surface of the patient's skin 14 (see FIG. 15B). For some such embodiments, the adhesive layer 153 may be covered with a peel away type covering 155 that is removed just prior to actuation of the applicator trigger 146 and subsequent actuation of the applicator 134 and contact of the adhesive layer 153 with the outer surface of the patient's skin 14. For some embodiments, the upper surface of the boss 124 of the packaging base may serve as the covering of the adhesive layer or any other such suitable configuration such that as the mount bracket 118 is removed from the boss 124, the adhesive layer 153 on the bottom surface 152 of the mount bracket 118 becomes automatically exposed and ready to adhere to the outer surface of the patient's skin 14. Such a configuration may save the end user from the additional step of removing a separate covering 155 thus making such an embodiment more user friendly and convenient.

In some instances, it may be desirable to include vent holes or fenestrations in the body of the base plate 119 of the mount bracket 118 in order to increase deployment velocity and reduce air drag and air entrapment/buffering between the bottom surface 152 of the mount bracket and the outside surface of the patient's skin during rapid downward translation of the mount bracket 118 towards the patient's skin 14 during deployment. It may also be desirable to minimize the weight of the mount bracket 118 overall in order to achieve a desired deployment velocity of the mount bracket 118 onto and into the patient's skin 14.

For some embodiments, the bottom surface 152 of the mount bracket 118 may be flat or substantially flat in profile. For some embodiments, the bottom surface 152 of the mount bracket may have a curved profile in order to better conform to certain outer profiles of the patient's anatomy at desired deployment sites. In some cases, the bottom surface of the mount bracket 118 may have a generally concave profile with a radius of curvature of about 50 mm to about 200 mm.

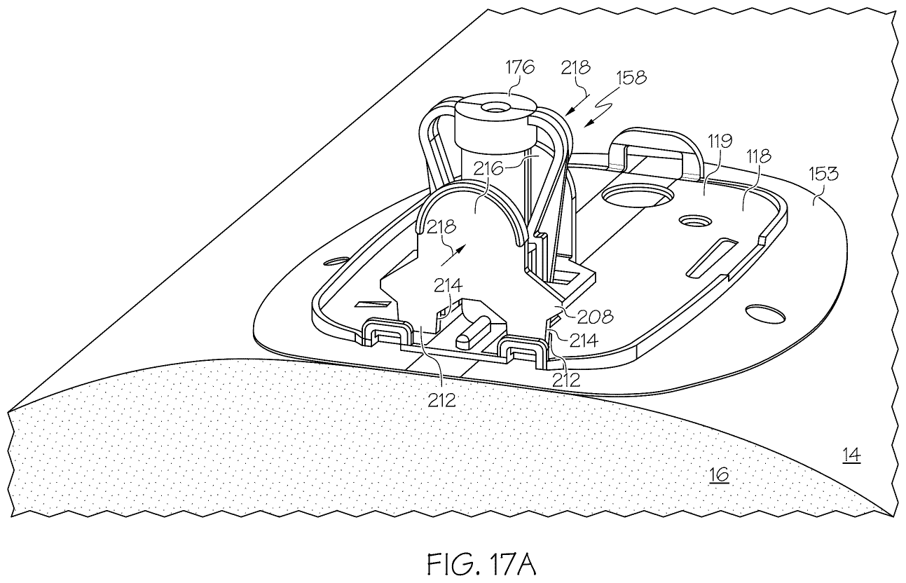

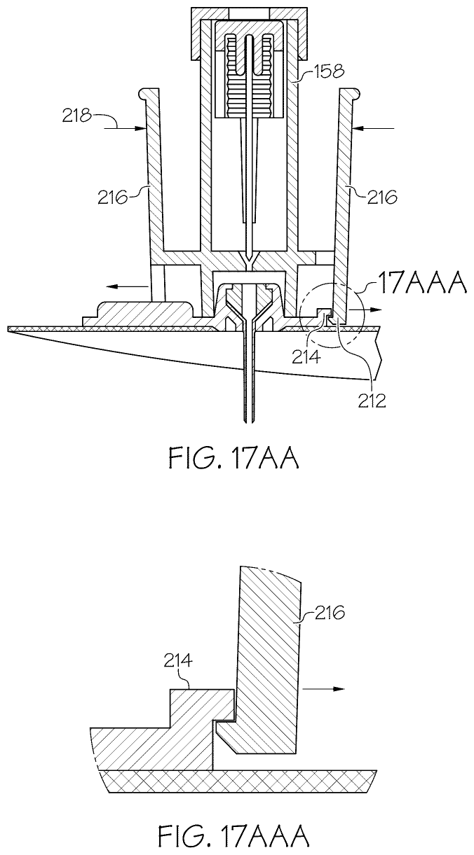

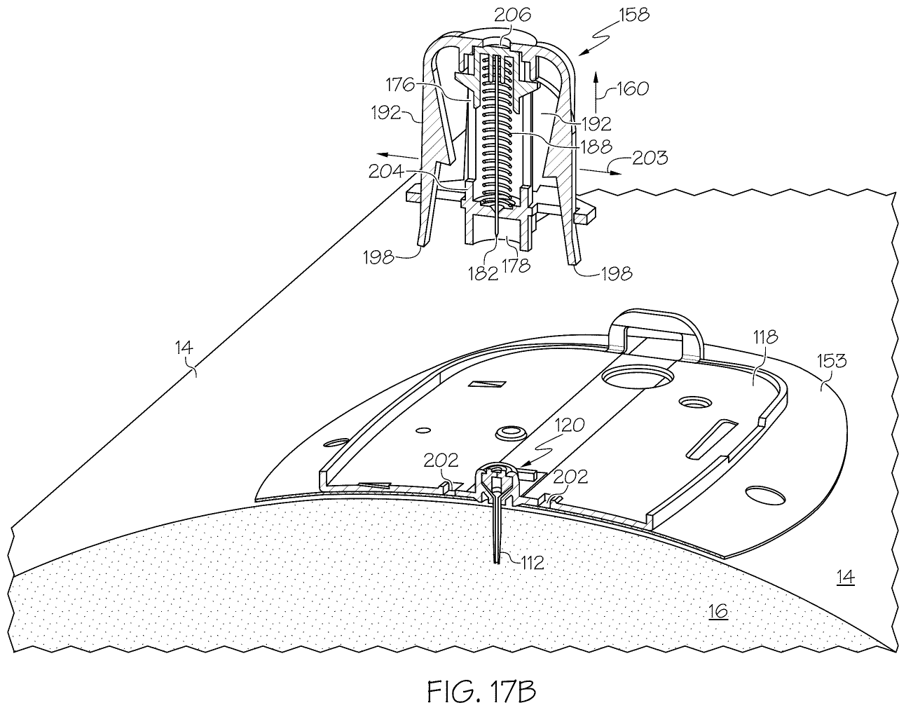

FIG. 17 shows the mount bracket 118 deployed onto the outer surface of the patient's skin 14 with the applicator 134 having been removed from the mount bracket 118 as indicated by the upward arrow 154 of FIG. 16 and with the cannula 112 and associated needle 144 disposed within and extending distally from an inner lumen of the cannula 112. Thereafter, the needle 144 may be removed from the inner lumen of the cannula 112 In some cases, the needle 144 may be removed by proximally withdrawing the needle cap assembly 158 and proximally withdrawing the needle cap assembly 158 and associated needle 144 upward and away from a bracket port 156 of the integrated infusion set 120 of the mount bracket 118. For some embodiments, the needle cap assembly may include an optional configuration that automatically withdraws the needle 144 into a needle receptacle 176 upon removal of the needle cap assembly 158 from the mount bracket 118. In some cases, such a needle receptacle 176 may include a needle cavity 178 which is configured to surround the needle 144 so as to isolate the needle 144, and particularly the sharpened distal tip 182 of the needle 144, and prevent user contact with the sharpened distal tip and also facilitate safe and convenient disposal of the used needle cap assembly 158.

An embodiment of such a needle cap assembly is shown in section in FIG. 17. The needle cap assembly 158 includes the needle 144 and a needle carrier 184 secured to a proximal end 186 of the needle 144. The needle carrier 184 is disposed within the needle receptacle 176 with a needle spring 188 which is shown in a compressed state and which is operatively secured between the needle receptacle 176 and the needle carrier 184. The needle spring 188 is arranged such that the needle spring 188 is configured to apply an upward proximal force onto the needle carrier 184 and needle 144 when released from the compressed state. The needle carrier 184 is held in place by a pair of needle spring retainers 192 with a retainer latch surface 194 of the needle spring retainers 192 releasably engaged with a corresponding and opposed carrier latch surface 196 of the needle carrier 184.

For some embodiments, the needle spring retainers 192 may have an elastic resilient property with an outward bias, indicated by arrows 193 of FIG. 17, that would leave the retainer latch surface 194 in a position that is not engaged with the corresponding carrier latch surface 196 when the needle spring retainers 192 are in a relaxed uncompressed state. In some cases, the retainer latch surface 194 may be held in an engaged state by the carrier latch surface 196 with the needle spring retainers 192 in a compressed state as shown in FIG. 17 by inwardly constraining end portions 198 of the needle spring retainers 192 with corresponding pockets 202, or any other suitable structure, on an element such as the base 119 of the mount bracket 118 as shown. For some embodiments, the needle carrier 184 may be disposed within a bore 204 of the needle receptacle 176. The bore 204 may be configured to restrict the translation of the needle carrier to a single degree of freedom along a longitudinal axis thereof. In some cases, the needle cavity 178 or portions thereof may include the bore 204 and the bore 204 may also include a proximal stop surface 206 to limit proximal translation of the needle carrier 184.

A perspective view of a needle cap assembly embodiment 158 is shown in FIG. 17A wherein the needle cap assembly 158 is releasably secured to the mount bracket 118 by a pair of the needle cap assembly retainers 208. The needle cap assembly retainers 208 are shown with clip ends 212 thereof releasably engaged with corresponding clip end receptacles 214 of the mount bracket 118. The needle cap assembly retainers 208 are held in such an engaged state with the clip ends 212 releasably coupled to the clip end receptacles 214 by an inward elastic bias of the clip ends 212. The clip ends 212 of the needle cap assembly retainers 208 may be disengaged from the clip end receptacles 214 of the mount bracket 118 by squeezing a proximal portion 216 of the needle cap assembly retainers 208, as indicated by arrows 218 in FIG. 17A, so as to overcome the inward elastic bias of the clip ends 212 and disengage them from the respective clip end receptacles 214.

For such embodiments, disengagement of the needle cap assembly 158 from the mount bracket 118 may include first releasing the needle cap assembly retainers 208 from the mount bracket 118 by, in some cases, squeezing the proximal portions 216 of the needle cap assembly retainers 208 and thereby releasing the engagement between the clip ends 212 of the needle cap retainers 208 and the clip end receptacles 214 of the mount bracket 118 as shown by arrows 218. Once the clip ends 212 are released from the clip end receptacles 214, the grip provided by squeezing the proximal portions 216 may then be used to proximally retract the needle cap assembly 158 from the mount bracket 118.

As the needle cap assembly 158 is being proximally retracted, the end portions 198 of the needle spring retainers 192 are also proximally withdrawn from the respective pockets 202 of the base 119 of the mount bracket 118 thereby releasing the constraint and allowing the outward bias of the needle spring retainers 192 to move them outwardly away from each other as indicated by arrows 203 in FIGS. 17B and 18. This outward translation of the needle spring retainers 192 and corresponding retainer latch surfaces 194 then disengages the retainer latch surface 194 of the needle spring retainers 192 from the corresponding and opposed carrier latch surface 196 of the needle carrier 184 which releases the compressed constraint on the needle spring 188. Thereafter, the release of the stored energy of the needle spring 188 then pushes the needle carrier 184 and needle 144 upwardly and proximally into the bore 204 of the needle receptacle 176 with the sharpened distal end 182 of the needle 144 drawn within the needle cavity 178 as shown in FIGS. 17B and 18.

FIG. 18 also shows the mount bracket 118 disposed on the outer surface of the patient's skin 14 with the needle 144 and needle cap assembly 158 removed from the inner lumen of the cannula and the bracket port 156 of the of the integrated infusion set of the mount bracket 118. The withdrawal of the needle 144 and associated needle cap assembly 158 is indicated by the upward arrow 160 of FIGS. 17B and 18. Withdrawal of the needle 144 and associated needle cap assembly 158 leaves the bracket port 156, and, in particular, the inner lumen of the cannula 112 disposed in fluid communication with the patient's tissue 16 beneath the outer surface of the patient's skin 14. In some instances, the cannula may extend from the bottom surface 152 of the mount bracket 118 by a length of about 4 mm to about 10 mm, more specifically, about 5 mm to about 7 mm.

It should be noted that in some cases the needle 144 is configured to provide the cannula 112 with a rigid tissue penetrating core structure so that the hollow cannula 112 may be made, in some instances, from a soft compliant material such as a polymer or the like which may be more comfortable for the patient after deployment and during use. The needle may thus be made from a high strength rigid material such as stainless steel, nitinol or the like and may include a sharpened distal tip which is configured for tissue penetration. The needle 144 may also have a generally straight configuration to facilitate tissue penetration in a linear motion along a longitudinal axis 145 thereof. In some cases, the sharpened distal tip of the needle 144 may include a trocar like faceted configuration. As discussed above, for some embodiments, the needle 144 may also be configured to automatically withdraw into a needle chamber (not shown) of the needle cap 158 upon withdrawal of the needle 144 from the cannula 112 and withdrawal of the needle cap 158 from the mount bracket 118. Such a configuration may be useful in order to protect the end user from the sharpened distal tip of the needle 144 and facilitate disposal of the needle cap 158 and needle 144 assembly after use.

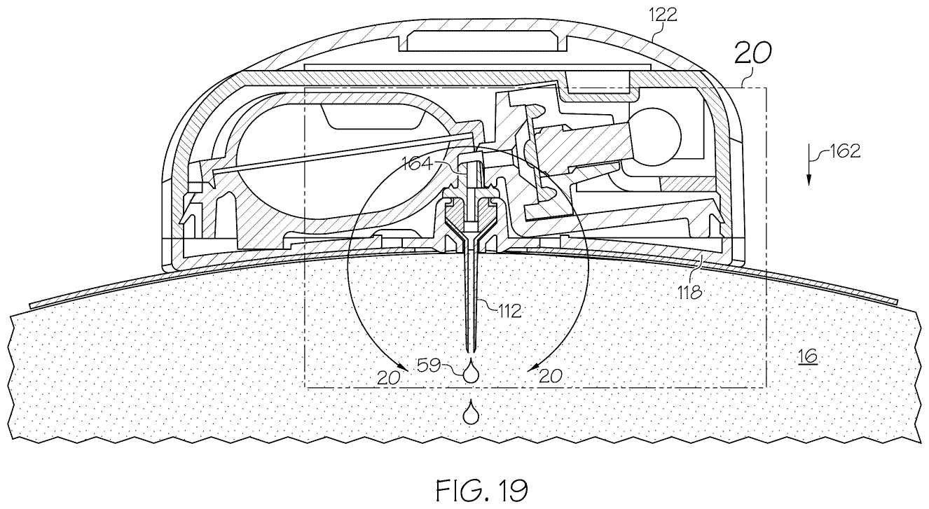

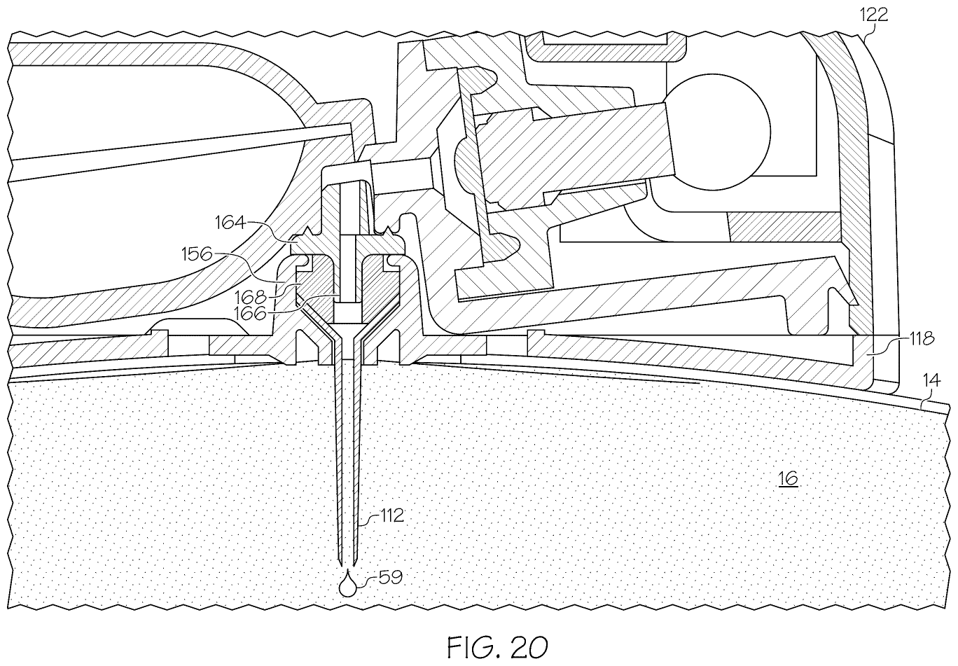

FIG. 19 shows the mount bracket 118 and cannula 112 thereof operatively coupled to the infusion pump assembly 122. The infusion pump assembly 122 has been coupled to the mount bracket 118 (as indicated by the downward arrow 162 in FIGS. 18B and 19) with a pump port 164 of the infusion pump assembly 122 operatively coupled to the bracket port 156 such that the pump port 164 is in fluid communication with the bracket port 156 in a configuration suitable for delivery of therapeutic fluid 59 from the infusion pump assembly 122 to the patient's body 16 including the patient's skin 14 and adjacent subdermal tissue. For some embodiments, the infusion pump assembly 122 may be operatively coupled to the mount bracket 118 by merely pressing the infusion pump assembly 122 down onto the mount bracket 118. For some such embodiments, the infusion pump assembly 122 and mount bracket 118 may be configured to mechanically couple together during such an approximation (such as by a snap fit or the like) and also automatically couple the pump port 164 to the bracket port 156 in fluid communication with each other as the needle 166 of the pump port 164 penetrates or otherwise operatively couples to the septum 168 of the bracket port 156 (as shown in FIG. 20) during the same simple movement. Such a configuration reduces the number of components required to be managed by the end user and simplifies the deployment process of the medical pump system 110 overall.

FIG. 20 illustrates an enlarged view of the pump port 164 coupled to the bracket port 156 and showing the hollow needle 166 of the pump port 164 disposed into and coupled with the septum 168 of the bracket port 156 providing sealed fluid communication between an inner lumen of the hollow needle 166 of the pump port 164 and the inner lumen of the cannula 112 of the mount bracket 118. This allows a sealed flow of therapeutic fluid 59 from the pump chamber (not shown) of the infusion pump assembly 122 through the hollow needle 166 of the pump port 164 and into an interior volume of the bracket port 156 and thereafter through the inner lumen of the cannula 112 and then out a distal port of the cannula 112 and into a subdermal region of the patient's tissue 16 as shown in FIG. 20. In some cases, the cannula 112 may include a hollow tubular structure that may be made from a flexible material including polymers such as polyurethane, nylon, PTFE or the like. In some cases, the cannula 112 may also be made from a biocompatible high strength rigid material such as stainless steel, nitinol or the like.

Once the use of an initial infusion pump assembly 122 has been completed, the initial infusion pump assembly 122 may be removed from the mount bracket 118 with the septum of the bracket port 156 self-sealing as the hollow needle 166 of the pump port 164 is withdrawn therefrom. Thereafter, a new infusion pump assembly 122 may be operatively coupled to the mount bracket 118 as described above or the mount bracket may be removed from the patient 16 by mechanically pulling the mount bracket 118 upwards away from the outer surface of the patient's skin 14. As the mount bracket 118 is removed from the patient, the cannula 112 will contemporaneously be withdrawn from the patient's body 16 and the delivery channel in the patient's skin 14 allowed to heal. In cases where the bottom surface 152 of the mount bracket 118 includes an adhesive layer, removal of the mount bracket 118 from the outer surface of the patient's skin 14 will require a breaking of the adhesive bond between the adhesive layer and the outer surface of the patient's skin 14. In addition to switching out a new infusion pump assembly 122 for an a previously deployed infusion pump assembly 122, a deployed infusion pump assembly 122 may be temporarily removed from the mount bracket 118 as noted above and then re-deployed onto the mount bracket 118 at a later time. Such temporary removal of a deployed infusion pump assembly 122 may be carried out to facilitate activities of a user such as swimming, bathing or the like.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this disclosure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A medical pump system, comprising:
a mount bracket including a base plate and an integral infusion set secured to the base plate which includes a cannula having an inner lumen and a bracket port in fluid communication with the inner lumen of the cannula;
an applicator that is configured to releasably engage the mount bracket and deploy the mount bracket away the from the applicator in a motion parallel to a longitudinal axis of the cannula, the applicator comprising an applicator driver and an applicator latch configured to releasably engage the mount bracket with the applicator driver in a restrained state;
an applicator trigger configured to release the applicator latch; and
a medical pump including a pump port that is configured to be operatively coupled in fluid communication to the bracket port.

2. The medical pump system of claim 1 wherein the entire outer peripheral contour of the mount bracket is disposed between a bottom surface of the medical pump and the patient's skin.

3. The medical pump system of claim 1 further comprising an adhesive layer disposed on a bottom surface of the mount bracket.

4. The medical pump system of claim 1 wherein a bottom surface of the base plate comprises a curved profile.

5. The medical pump system of claim 4 wherein the curved profile of the bottom surface of the mount bracket has a radius of curvature of about 50 mm to about 200 mm.

6. The medical pump system of claim 1 wherein the cannula extends from a bottom surface of the mount bracket by a length of about 4 mm to about 10 mm.

7. The medical pump system of claim 1 wherein the pump port comprises a pump port needle which is configured to penetrate a septum of the bracket port with an inner lumen of the pump port needle in fluid communication with the inner lumen of the cannula.

8. The medical pump system of claim 1 wherein the integral infusion set further comprises a needle that is rigid with a distal tip and core structure configured to penetrate tissue and that is slidingly disposed within the inner lumen of the cannula.

9. The medical pump system of claim 8 further comprising a needle cap which is secured to a proximal end of the needle and which is releasably secured to the bracket port.

10. The medical pump system of claim 8 wherein the cannula comprises a soft compliant material.

11. The medical pump system of claim 1 wherein the applicator driver comprises a spring.

12. The medical pump system of claim 11 wherein the spring of the applicator driver comprises a coil spring.

13. The medical pump system of claim 1 wherein the medical pump is configured to be releasably coupled and secured to the mount bracket.

14. A medical pump system, comprising:
a mount bracket, including
a base plate,
an integral infusion set secured to the base plate which includes a cannula having an inner lumen and a bracket port in fluid communication with the inner lumen of the cannula,
a needle cap assembly which is releasably secured in fixed relation to the integral infusion set and which includes a needle extending through the inner lumen of the cannula;
an applicator that is configured to releasably engage the mount bracket and deploy the mount bracket away the from the applicator in a motion parallel to a longitudinal axis of the cannula, the applicator comprising an applicator driver and an applicator latch configured to releasably engage the mount bracket with the applicator driver in a restrained state;
an applicator trigger configured to release the applicator latch; and
a medical pump including a pump port that is configured to be operatively coupled in fluid communication to the bracket port and releasably secured to the base plate.

15. The medical pump system of claim 14 wherein the needle cap assembly is configured to automatically withdraw the needle into a needle receptacle of the needle cap assembly upon removal of the needle cap assembly from the mount bracket.

16. The medical pump system of claim 15 wherein the needle cap assembly includes a needle carrier secured to a proximal end of the needle, the needle carrier being slidably disposed within the needle receptacle, a needle spring operatively coupled to the needle carrier and configured to impart a proximal retractive force on the needle carrier and needle and at least on needle spring retainer configured to hold the needle spring in a restrained state and be deployed releasing the needle spring upon retraction of the needle cap assembly from the mount bracket.

17. The medical pump system of claim 14 wherein the needle cap assembly is releasably secured in fixed relation to the integral infusion set with at least one needle cap assembly retainer.

18. A mount bracket for interfacing with a patient, comprising
a base plate;
an integral infusion set secured to the base plate within an outer contour of the base plate, the integral infusion set including
a bracket port which is configured to be operatively coupled in fluid communication to a medical pump, and
a cannula which extends outwardly from a bottom surface of the base plate and which includes an inner lumen disposed in fluid communication with the bracket port; and
an adhesive layer disposed on the bottom surface of the base plate which is configured to temporarily adhere to the skin of a patient with a distal tip of the cannula disposed beneath an outer surface of the patient's skin.

19. The mount bracket of claim 18 wherein a longitudinal axis of the cannula is perpendicular to an adjacent bottom surface of the base plate.

20. The mount bracket of 18 wherein a bottom surface of the base plate comprises a curved profile.

21. The mount bracket of claim 20 wherein the curved profile of the bottom surface of the mount bracket has a radius of curvature of about 50 mm to about 200 mm.

22. The mount bracket of claim 18 wherein the cannula extends from the bottom surface of the base plate by a length of about 4 mm to about 10 mm.

23. The mount bracket of claim 18 wherein the integral infusion set further comprises a needle that is rigid with a distal tip and core structure configured to penetrate tissue and that is slidingly disposed within the inner lumen of the cannula.

24. The mount bracket of claim 23 further comprising a needle cap which is secured to a proximal end of the needle and which is releasably secured to the bracket port.

25. The mount bracket of claim 23 wherein the cannula comprises a soft compliant material.

26. The mount bracket of claim 18 wherein the base plate is configured to releasably secure a medical pump thereto.
